(19) United States
(12) Patent Application Publication
(10) Pub. No.: US 2023/0083251 A1
(43) Pub. Date: Mar. 16, 2023

(54) SYSTEM AND METHOD FOR A WEARABLE MONITORING DEVICE

(71) Applicant: MAE Holdings LLC, Marietta, GA (US)

(72) Inventor: Michael Justin Runager, Marietta, GA (US)

(21) Appl. No.: 17/473,420

(22) Filed: Sep. 13, 2021

(51) Int. Cl. G04G 99/00; G04G 9/00; G04G 13/02; G06F 3/01

(52) U.S. Cl. CPC G04G 99/006; G04G 9/0005; G04G 9/0017; G04G 13/021; G06F 3/017

(57) ABSTRACT

The present disclosure is directed at a system that comprises at least one detection based unit configured to detect a decibel level associated with at least one space in a building and generate an alert when a predetermined decibel level is detected and wirelessly transmit that alert to a client device via a main base unit.

### CROSS-REFERENCE TO RELATED APPLICATIONS

This application claims priority to U.S. Provisional Application No. 63/079,829 filed on Sep. 17, 2020, the entirety of which is herein incorporated by reference.

### FIELD

The present disclosure generally relates to the technical field of monitoring devices. More specifically, the present disclosure is directed to a system for monitoring a space according to a predetermined audio decibel level via a client device.

### BACKGROUND

For light sleepers, the biggest challenge is getting back to sleep after becoming awake. This is especially true for parents of newborn or infant children. Baby monitors that are wirelessly connected to a device in the kids room either produce constant noise or turn on abruptly when crying is detected. For light sleepers this means any cry, yell, or audible sound may wake up a parent or individual.

Furthermore, traditional baby monitoring systems do not allow for free movement around the house and surrounding areas. Specifically, the monitor is often in a semi-fixed location with a finite communication range from the device located in the kid’s room. The monitor is also cumbersome and large making it difficult to move around constantly. Finally, the monitor needs a constant charge which often requires co-locating the monitor near an available outlet for optimum use without worrying if the battery will degrade. Battery life on conventional baby monitors is poor and declines over time and the lack of portability of such devices makes monitoring difficult when disconnected from a constant power source.

A further impediment to conventional monitoring systems is the ability to perform tasks without constantly being in close proximity to the monitor. Current monitoring systems require a user to be in close proximity to the monitor, such that any noises or alerts that may indicate a potential issue can only be received based on being able to hear or see the monitor. Communication coverage for the monitor to maintain reception with the device located in the room is limited. Finally, an activity with significant background noise will make alerts or noises useless because the audio level of the monitor is limited.

According to various embodiments, the present disclosure is directed to a wireless wearable device that vibrates when a predetermined level of noise is reached. In some embodiments, the wearable device communicates with a base that is plugged into a wall socket and includes a decibel reader for detecting a level of noise and subsequently alerts the wearable device, either via wireless communications. In some embodiments, each base is identified or named by the user and the wearable device will identify which base is picking up the noise. In various embodiments, a plurality of base units having audible detection capability are communicably coupled to a main base unit. In this embodiment, the main base unit is communicably coupled to a plurality of wearable devices such that alerts (i.e., messages, vibrations, alarms, etc.) may be communicated to a user of the wearable device. In some embodiments, the alerts are transmitted to a client device of the user (i.e., smart phone, etc.). In this embodiment, the system may communicate audible alerts to the smart phone and/or a wearable device. As will be further identified, communications are not limiting and the system is configured to send alerts to a plurality of client devices of the user.

The wireless nature of this wearable device will allow someone to move freely, while utilizing a vibration or message to alert the person wearing the device if there is noise in the room where the base is active.

In some embodiments, the system is configured to identify motion and/or specific audible commands to alert a user of specific events associated with the motion and/or specific audible commands. As will be explained in further detail, the system may be configured to be programmed or customized which advantageously allows the user of the system to program alerts to specific events associated with an audible decibel level, motion, and/or specific command.

### BRIEF DESCRIPTION OF THE DRAWINGS

Various aspects of the present disclosure will be or become apparent to one with skill in the art by reference to the following detailed description when considered in connection with the accompanying exemplary non-limiting embodiments, in which:

FIG. 1A illustrates one example of a system in accordance with some embodiments of the present disclosure.

FIG. 1B illustrates one example of an architecture of a mobile device in accordance with some embodiments of the present disclosure.

FIG. 2 illustrates an example system schematic with system components in accordance with some embodiments of the present disclosure.

FIG. 3 is a flow diagram illustrating an example in accordance with some embodiments of the present disclosure.

FIG. 4 is a flow diagram illustrating an example detection method in accordance with some embodiments of the present disclosure.

FIGS. 5A and 5B are wearable monitors in accordance with some embodiments of the present disclosure.

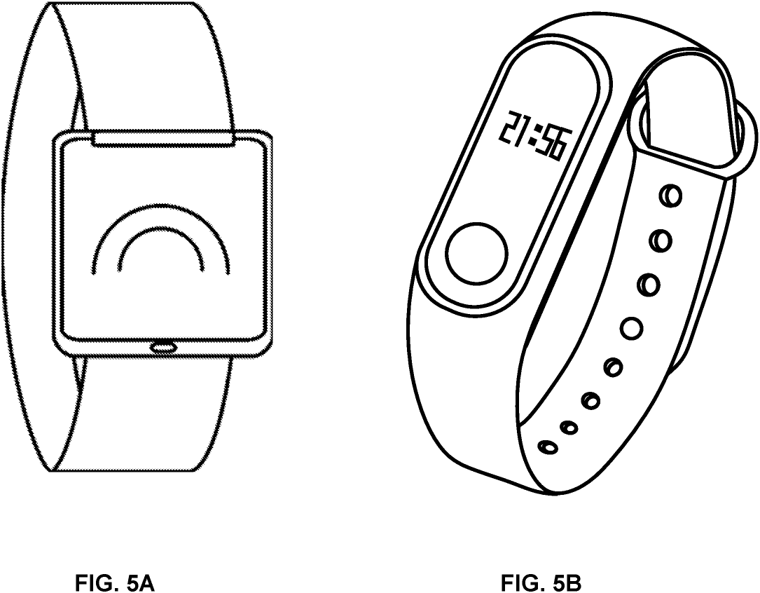

FIG. 6 is a decibel reader in accordance with some embodiments of the present disclosure.

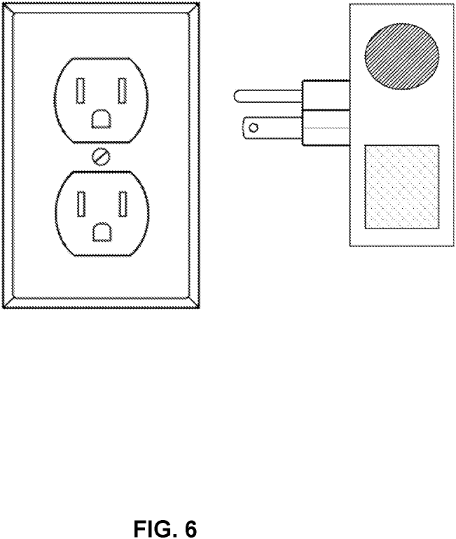

### SUMMARY

According to various embodiments of the present disclosure, a system is disclosed. The system comprises at least one detection based unit configured to detect a decibel level associated with at least one space in a building and generate an alert when a predetermined decibel level is detected. The system further comprises at least one main base unit configured to be communicatively coupled to the at least one detection base unit and wirelessly receive and transmit the generated alert; and at least one client device configured to receive the transmitted alert and identify information associated with the alert relating to the space within the building that corresponds to the associated detection base unit that generated the alert.

According to various embodiments of the present disclosure, a system is disclosed. The system comprises at least one detection based unit configured to detect a decibel level associated with at least one space in a building and generate an alert when either a predetermined decibel level or preprogrammed audio phrase is detected. The system further comprises at least one main base unit configured to be communicatively coupled to the at least one detection base unit and wirelessly receive and transmit the generated alert; and at least one client device configured to receive the transmitted alert and identify information associated with the alert relating to the space within the building that corresponds to the associated detection base unit that generated the alert.

According to various embodiments of the present disclosure, a method is disclosed. The method comprises the steps of detecting a decibel level by at least one detection base unit associated with at least one space in a building; generating an alert when a predetermined decibel level is detected; receiving the generated alert wirelessly by at least one main base unit; transmitting the generated alert from the at least one main base unit to at least one client device; and identifying information associated with generated alert at the client device associated with the space within the building that corresponds to the respective detection base unit that generated alert.

### DETAILED DESCRIPTION

This description of the exemplary embodiments is intended to be read in connection with the accompanying drawings, which are to be considered part of the entire written description. The use of the singular includes the plural unless specifically stated otherwise. The use of “or” means “and/or” unless stated otherwise. Furthermore, the use of the term “including,” as well as other forms such as “includes” and “included,” is not limiting. In addition, terms such as “element” or “component” encompass both elements and components comprising one unit, and elements and components that comprise more than one subunit, unless specifically stated otherwise. Additionally, the section headings used herein are for organizational purposes only, and are not to be construed as limiting the subject matter described.

The following description is provided as an enabling teaching of a representative set of examples. Many changes can be made to the embodiments described herein while still obtaining beneficial results. Some of the desired benefits discussed below can be obtained by selecting some of the features discussed herein without utilizing other features. Accordingly, many modifications and adaptations, as well as subsets of the features described herein are possible and can even be desirable in certain circumstances. Thus, the following description is provided as illustrative and is not limiting.

As used herein, use of a singular article such as “a,” “an” and “the” is not intended to exclude pluralities of the article’s object unless the context clearly and unambiguously dictates otherwise

### System Overview

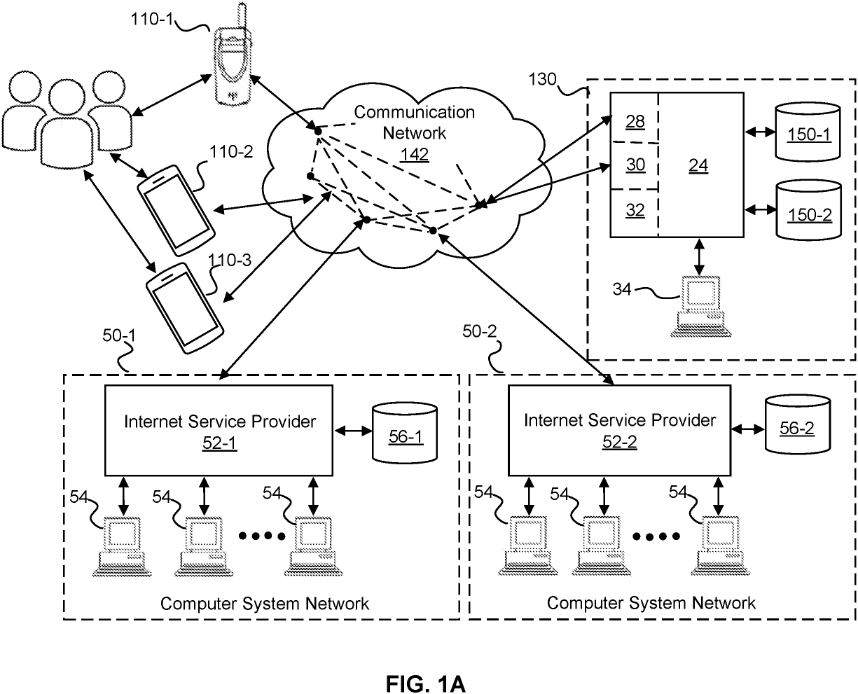

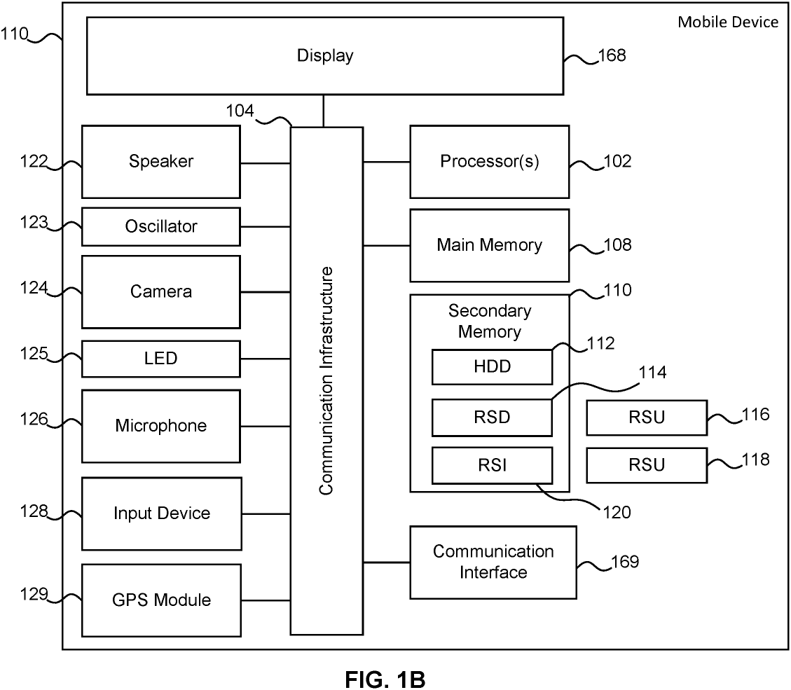

In various embodiments, the monitoring system may interact with client devices for identifying alerts associated with detection devices in the system. FIG. 1A depicts one example of a system 100 in which a plurality of client devices 110-1, 110-2, and 110-3 (collectively “client devices 110”) are connected via communication network 142 to one or more computer system networks 50-1, 50-2 (“computer networks 50”), and to management server 130. Communication network 142 may be a wide area network (“WAN”), a local area network (“LAN”), personal area network (“PAN”), or the like. In one embodiment, communication network 142 is the Internet and client devices 110 are online. “Online” may mean connecting to or accessing source data or information from a location remote from other devices or networks coupled to communication network 142.

Management server 130 includes a processing unit 24 coupled to one or more data storage units 150-1, 150-2 (collectively referred to as “database management system 150” or “DBMS 150”). The processing unit 24, in some embodiments is configured to provide front-end graphical user interfaces (“GUI”) 28, and a back-end or administrative graphical user interface or portal 32 to one or more remote computers 54 or to one or more local computers 34. In some embodiments, an interface 30, described in further detail below, is provided that accesses management server 130 via GUI 28. The GUIs can take the form of, for example, a webpage that is displayed using a browser program local to remote computers 54 or to one or more local computers 34. It is understood that the system 100 may be implemented on one or more computers, servers, or other computing devices. In some embodiments, the GUI may be displayed on client devices 110 via a software application. For example, system 100 may include additional servers programmed or partitioned based on permitted access to data stored in DBMS 150. As used herein, “portal” is not limited to general-purpose Internet portals, such as YAHOO! ™ or GOOGLE ™ but also includes GUIs that are of interest to specific, limited audiences and that provide the party access to a plurality of different kinds of related or unrelated information, links and tools as described below. “Webpage” and “website” may be used interchangeably herein.

Remote computers 54 may be part of a computer system network 50-1, 50-2 and gain access to communication network 142 through an Internet service provider (“ISP”) 52-1, 52-2 (“ISPs 52”). Computer system networks 50-1 and 50-2 may comprise, in some embodiments, data storage or DBMS 56-1 and 56-2, respectively. Client devices 110 may gain access to communications network 142 through a wireless cellular communication network, a WAN hotspot, or through a wired or wireless connection with a computer as will be understood by one skilled in the art. As will be described below, a user may use remote computers 54 and/or client devices 110 to gain access to system 100.

In one embodiment, client devices 110 includes any mobile device capable of transmitting and receiving wireless signals. Examples of mobile instruments include, but are not limited to, mobile or cellular phones, smart phones. personal digital assistants (“PDAs”), laptop computers, tablet computers, music players, wearable peripheral devices, and e-readers, to name a few possible devices.

FIG. 1B is a block diagram of one example of an architecture of client device 110. As shown in FIG. 1B, client device 110 includes one or more processors, such as processor(s) 102. Processor(s) 102 may be any central processing unit (“CPU”), microprocessor, micro-controller, or computational device or circuit for executing instructions. Processor(s) are connected to a communication infrastructure 104 (e.g., a communications bus, cross-over bar, or network). Various software embodiments are described in terms of this exemplary client device 110. After reading this description, it will be apparent to one of ordinary skill in the art how to implement the method using client devices 110 that include other systems or architectures. One of ordinary skill in the art will understand that computers 34, 54 may have a similar and/or identical architecture as that of client devices 110. Put another way, computers 34, 54 can include some, all, or additional functional components as those of the client device 110 illustrated in FIG. 1B.

In various embodiments, client device 110 includes a display 168 that displays graphics, video, text, and other data received from the communication infrastructure 104 (or from a frame buffer not shown) to a user (e.g., a subscriber, commercial user, back-end user, or other user). Examples of such displays 168 include, but are not limited to, LCD screens, OLED display, capacitive touch screen, and a plasma display, to list only a few possible displays. Client device 110 also includes a main memory 108, such as a random access (“RAM”) memory, and may also include a secondary memory 110. Secondary memory 110 may include a more persistent memory such as, for example, a hard disk drive (“HDD”) 112 and/or removable storage drive (“RSD”) 114, representing a magnetic tape drive, an optical disk drive, solid state drive (“SSD”), or the like. In some embodiments, removable storage drive 114 reads from and/or writes to a removable storage unit (“RSU”) 116 in a manner that is understood by one of ordinary skill in the art. Removable storage unit 116 represents a magnetic tape, optical disk, or the like, which may be read by and written to by removable storage drive 114. As will be understood by one of ordinary skill in the art, the removable storage unit 116 may include a tangible and non-transient machine readable storage medium having stored therein computer software and/or data.

In some embodiments, secondary memory 110 may include other devices for allowing computer programs or other instructions to be loaded into client device 110. Such devices may include, for example, a removable storage unit (“RSU”) 118 and a corresponding interface (“RSI”) 120. Examples of such units 118 and interfaces 120 may include a removable memory chip (such as an erasable programmable read only memory (“EPROM”)), programmable read only memory (“PROM”)), secure digital (“SD”) card and associated socket, and other removable storage units 118 and interfaces 120, which allow software and data to be transferred from the removable storage unit 118 to client device 110.

In various embodiments, client device 110 may also include a speaker 122, an oscillator 123, a camera 124, a light emitting diode (“LED”) 125, a microphone 126, an input device 128, an accelerometer (not shown), and/or a global positioning system (“GPS”) module 129. Examples of input device 128 include, but are not limited to, a keyboard, buttons, a trackball, or any other interface or device through which a user may input data. In some embodiment, input device 128 and display 168 are integrated into the same device. For example, display 168 and input device 128 may be touchscreen through which a user uses a finger, pen, and/or stylus to input data into client device 110.

Client device 110 also includes one or more communication interfaces 169, which allows software and data to be transferred between client device 110 and external devices such as, for example, another client device 110, a computer 34, 54 and other devices that may be locally or remotely connected to system 100. Examples of the one or more communication interfaces 169 may include, but are not limited to, a modem, a network interface (such as an Ethernet card or wireless card), a communications port, a Personal Computer Memory Card International Association (“PCMCIA”) slot and card, one or more Personal Component Interconnect (“PCI”) Express slot and cards, or any combination thereof. The one or more communication interfaces 169 may also include a wireless interface configured for short range communication, such as near field communication (“NFC”), Bluetooth, or other interface for communication via another wireless communication protocol. As briefly noted above, one of ordinary skill in the art will understand that computers 34, 54 and portions of system 100 may include some or all components of client device 110.

Software and data transferred via the one or more communications interfaces 169 are in the form of signals, which may be electronic, electromagnetic, optical, or other signals capable of being received by communications interfaces 169. These signals are provided to communications interface 169 via a communications path or channel. The channel may be implemented using wire or cable, fiber optics, a telephone line, a cellular link, a radio frequency (“RF”) link, or other communication channels.

In this document, the terms “non-transitory computer program medium” and “non-transitory computer readable medium” refer to media such as removable storage units 116, 118, or a hard disk installed in hard disk drive 112. These computer program products provide software to client device 110. Computer programs (also referred to as “computer control logic”) may be stored in main memory 108 and/or secondary memory 110. Computer programs may also be received via the one or more communications interfaces 169. Such computer programs, when executed by a processor(s) 102, enable the client device 110 to perform the features of the method discussed herein.

In various embodiments, system 100 may be a computing environment including one or more client devices 110, management server 130, one or more software management modules 131, 132, 133, 134, and 135 (not shown), one or more software engines 136 and 137 (not shown), database connection interface 143 (not shown), database management system 150 (not shown), and a communication network 142 connecting various components of system 100. Although one client device 110 is described, any number of client devices may be present. In various embodiments, client device 110 is a user device capable of connecting to the Internet or similar network as will be described below.

In various embodiments, as shown in FIGS. 1A-1B, client device 110 may include a computing device such as a hashing computer, a personal computer, a laptop computer, a tablet computer, a notebook computer, a hand-held computer, a personal digital assistant, a portable navigation device, a mobile phone, a smart phone, a wearable computing device (e.g., a smart watch, a wearable activity monitor, wearable smart jewelry, and glasses and other optical devices that include optical head-mounted displays (OHMDs)), an embedded computing device (e.g., in communication with a smart textile or electronic fabric), or any other suitable computing device configured to store data and software instructions, execute software instructions to perform operations, and/or display information on a display device. Client device 110 may be associated with one or more users (not shown). For example, a user operates client device 110, causing it to perform one or more operations in accordance with various embodiments.

In some embodiments, client device 110 includes one or more tangible, non-transitory memories that store data and/or software instructions, and one or more processors configured to execute software instructions. Client device 110 may include one or more display devices that display information to a user and one or more input devices (e.g., keypad, keyboard, touchscreen, voice activated control technologies, or any other suitable type of known input device) to allow the user to input information to the client device. Client device 110 processor(s) may be any central processing unit (“CPU”), microprocessor, micro-controller, or computational device or circuit for executing instructions. Processor(s) are connected to a communication infrastructure (e.g., a communications bus, cross-over bar, or network). Various software embodiments are described in terms of this exemplary client device 110. After reading this description, it will be apparent to one of ordinary skill in the art how to implement the method using client device 110 that include other systems or architectures. One of ordinary skill in the art will understand that computers may have a similar and/or identical architecture as that of client device 110. Put another way, computers can include some, all, or additional functional components as those of the client device 110 illustrated in FIGS. 1A-1B.

In various embodiments, client device 110 also includes one or more communication interfaces 169, which allows software and data to be transferred between client device 110 and external devices such as, for example, another client device 110, a computer, management server 130, and other devices that may be locally or remotely connected to client device 110. Examples of the one or more communication interfaces may include, but are not limited to, a modem, a network interface (e.g., communication interface 169, such as an Ethernet card or wireless card), a communications port, a Personal Computer Memory Card International Association (“PCMCIA”) slot and card, one or more Personal Component Interconnect (“PCI”) Express slot and cards, or any combination thereof. The one or more communication interfaces 169 may also include a wireless interface configured for short range communication, such as near field communication (“NFC”), Bluetooth, or other interface for communication via another wireless communication protocol.

Software and data transferred via the one or more communications interfaces 169 are in the form of signals, which may be electronic, electromagnetic, optical, or other signals capable of being received by communications interfaces. These signals are provided to communications interface 169 via a communications path or channel. The channel may be implemented using wire or cable, fiber optics, a telephone line, a cellular link, a radio frequency (“RF”) link, or other communication channels.

In an embodiment where the system 100 or method is partially or entirely implemented using software, the software may be stored in a computer program product and loaded into client device 110 using removable storage drive, hard drive, and/or communications interface. The software, when executed by processor(s), causes the processor(s) to perform the functions of the method described herein. In another embodiment, the method is implemented primarily in hardware using, for example, hardware components such as application specific integrated circuits (“ASICs”). Implementation of the hardware state machine so as to perform the functions described herein will be understood by persons skilled in the art. In yet another embodiment, the method is implemented using a combination of both hardware and software.

Embodiments of the subject matter described in this specification can be implemented in a system 100 that includes a back end component, e.g., as a data server, or that includes a middleware component, e.g., an application server, or that includes a front end component (e.g., a client device 110) having a graphical user interface or a Web browser through which a user can interact with an implementation of the subject matter described is this specification, or any combination of one or more such back end, middleware, or front end components. The components of the system can be interconnected by any form or medium of digital data communication, (e.g., a communication network 142). Communications network 142 may include one or more communication networks or media of digital data communication. Examples of communication network 142 include a local area network (“LAN”), a wireless LAN, a RF network, a Near Field Communication (NFC) network, (e.g., a “WiFi” network), a wireless Metropolitan Area Network (MAN) connecting multiple wireless LANs, NFC communication link(s), and a wide area network (“WAN”), e.g., the Internet and combinations thereof. In accordance with various embodiments of the present disclosure, communications network 142 may include the Internet and any publicly accessible network or networks interconnected via one or more communication protocols, including, but not limited to, hypertext transfer protocol (HTTP) and HyperText Transfer Protocol Secured (HTTPS) and Secured Socket Layer/Transport Layer Security (SSL/TLS) and transmission control protocol/internet protocol (TCP/IP). Communications protocols in accordance with various embodiments also include protocols facilitating data transfer using radio frequency identification (RFID) communications and/or NFC. Moreover, communications network 142 may also include one or more mobile device networks, such as a GSM or LTE network or a PCS network, allowing a client device to send and receive data via applicable communications protocols, including those described herein. For ease of illustration, communication network 142 is shown as an extension of management server 130.

A client device 110 and server 130 are generally remote from each other and typically interact through a communication network 142. The relationship of client device 110 and management server 130 arises by virtue of computer programs running on the respective system components and having a client-server relationship to each other. System 100 may include a web/application server (not shown) in embodiments used to gain access to many services provided by management server 130.

In one aspect, client device 110 stores in memory one or more software applications that run on the client device and are executed by the one or more processors. In some instances, each client device stores software applications that, when executed by one or more processors, perform operations that establish communications with management server 130 (e.g., across communication network 142 via communication interface 169) and that obtain, from management server 130, information or data via database management system 150 in accordance with various embodiments.

In various embodiments, client device 110 may execute stored software application(s) to interact with management server 130 via a network connection. The executed software applications may cause client device 110 to communicate information. Stored software application(s) on client device 110 are configured to access webpages on the Internet or other suitable network based communication capable of interacting with communication network 142, as would be understood by one of ordinary skill in the art. For example, a user may access a user account on management server 130 via an Internet webpage. In this example, management server 130 is configured to render the Internet webpage for the user on client device 110. Alternatively, management server 130 may provide information to stored software application(s) on client device 110 via communication network 142. In this example, client device 110 will display information provided by management server 130 using a stored software application(s) graphical user interface display. In the example above, a respective user account may be associated with a developer, client user, or supervisor/monitoring authority as would be understood by one of ordinary skill in the art and described below.

According to various embodiments, system 100 includes database management system/storage 150 for managing and storing data, wayfinding service information, and other data maintained by the management server 130. The database management system and/or storage are referred to herein simply as DBMS 150 for convenience. DBMS 150 is communicatively coupled with various modules and engines as illustrated in FIGS. 1A-1B.

It should be understood that various forms of data storage or repositories can be used in system 100 that may be accessed by a computing system, such as hard drives, tape drives, flash memory, random-access memory, read-only memory, EEPROM storage, in-memory databases like SAP HANA, and so on, as well as any combination thereof. Stored data may be formatted within data stores in one or more formats, such as flat text file storage, relational databases, non-relational databases, XML, comma-separated values, Microsoft Excel files, or any other format known to those of ordinary skill in the art, as well as any combination thereof as is appropriate for the particular use. Data stores may provide various forms of access to the stored data, such as by file system access, network access, a SQL protocol (e.g., ODBC), HTTP, FTP, NES, CIFS, and so on, as well as any combination thereof.

According to various embodiments, client device 110 is configured to access DBMS 150 via management server 130. In various embodiments, DMBS 150 is configured to maintain a database schema. As will be described in further detail below, database schema 180 is configured to maintain a plurality of identifiers associated with specific categories of records. For example, database schema may be arranged to maintain identifiers in columns within DBMS 150. In this aspect, identifiers refer to specific information pertaining to the categories described above. Database schema 180 within DMBS 150 may be arranged or organized in any suitable manner within the system. Although the above described examples identify categorical identifiers, any number of suitable identifiers may be used to maintain records associated with the system described herein. In addition, a database schema may contain additional categories and identifiers not described above for maintaining record data in system 100. The database can also provide statistics and marketing information associated with users of system 100.

The database schema described above advantageously organizes identifiers in a way that permits the system to operate more efficiently. In some embodiments, categories of identifiers in the database schema increase efficiency by grouping identifiers with an associated management model of management server 130.

In various embodiments, management server 130 includes computing components configured to store, maintain, and generate data and software instructions. For example, management server 130 may include or have access to one or more processors 24, one or more servers (not shown) and tangible, non-transitory memory devices (e.g., local data store (in addition to DBMS 150)) for storing software or code for execution and/or additional data stores. Servers may include one or more computing devices configured to execute software instructions stored on to perform one or more processes in accordance with various embodiments. In some embodiments, DBMS 150 includes a server that executes software instructions to perform operations that provide information to at least one other component of computing environment 100, for example providing data to another data store or to third party recipients (e.g., banking systems, third party vendors, information gathering institutions, etc.) through a network, such as a communication network 142.

Management server 130 may be configured to provide one or more websites, digital portals, or any other suitable service that is configured to perform various functions of management server 130 components. In some embodiments, management server 130 maintains application programming interfaces (APIs) through which the functionality and services provided by server 130 may be accessed through one or more application programs executed by a client device 110. In various embodiments, management server 130 may provide information to software application(s) on client device 110 for display on a graphical user interface 168.

In some embodiments, management server 130 provides information to client device 110 (e.g., through the API associated with the executed application program). Client device 110 presents portions of the information to corresponding users through a corresponding respective graphical user interface 168 or webpage.

In various embodiments, management server 130 is configured to provide or receive information associated with services provided by management server 130 to client device 110. For example, client device 110 may receive information via communication network 142, and store portions of the information in a locally accessible store device and/or network-accessible storage devices and data stores (e.g., cloud-based storage). For example, client device 110 executes stored instructions (e.g., an application program, a web browser, and/or a mobile application) to process portions of stored data and render portions of the stored data for presentation to the respective user or users. Management server 130 may include additional servers (not shown) which may be incorporated as a corresponding node in a distributed network or as a corresponding networked server in a cloud-computing environment. Furthermore, servers may communicate via communication network 142 with one or more additional servers (not shown), that may facilitate the distribution of processes for parallel execution by the additional servers.

### Description of Monitoring System

Various embodiments of the present disclosure are directed to a wearable device that oscillates or causes vibrations triggered by a detection device that detects a predetermined decibel/volume level. Specifically, the wearable device may be actuated by a specific programmable phrase or audible sound (i.e., crying, etc.) at a sufficient audible level. The actuation of the wearable device may trigger a vibration and/or identify visually in which room the noise is occurring. Although a wearable device is described in this embodiment, a person of skill in the art would understand that any client device as previously described may be used to achieve the functions described herein.

In various embodiments, the wearable device is wirelessly connected to a base located in each room. As used herein, a base may refer to a variety of detection devices to include audible and motion detection devices. In some embodiments, the wearable device is portable or mobile and may be freely moved around the base located in a particular room without having to carry an additional monitor. For example, the wearable device may be worn outside of the structure and be configured to receive alerts related to a predetermined decibel/volume level, a programmable phrase, or motion. In various embodiments, the wearable device may be any client device previously described. For example, in some embodiments, the base is configured to generate an alert based on detecting movement, audible level, or audible phrase. In this embodiment, the base may communicate the associated alert to a client device (i.e., smart phone) via communication network 142.

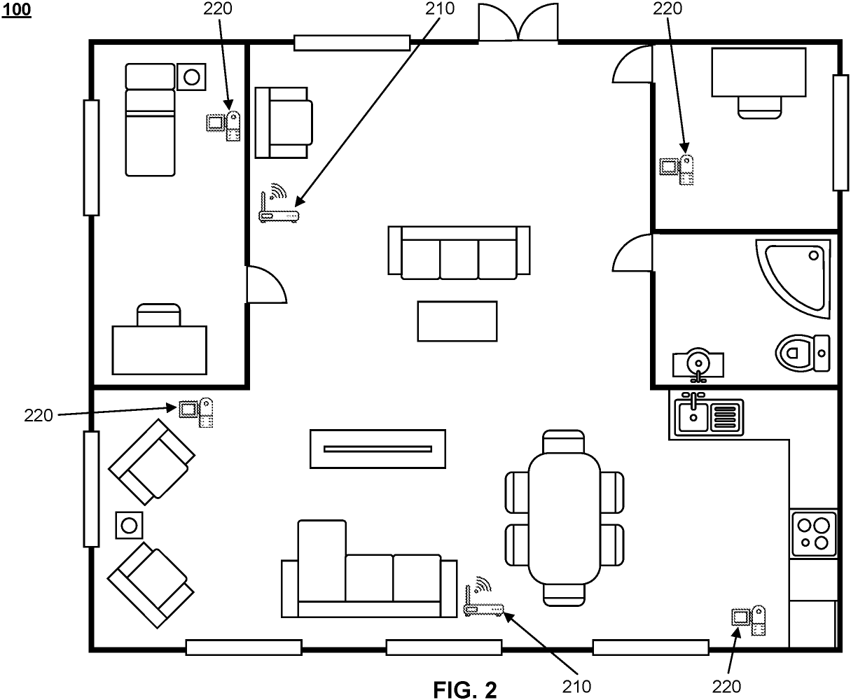

FIG. 2 illustrates an example system schematic with system components in accordance with some embodiments of the present disclosure. In various embodiments, as depicted in FIG. 2, system 100 includes a main base unit 210. In some embodiments, system 100 further includes a plurality of base units 220 and client devices 110. In various embodiments, main base unit 210 is configured to be communicably coupled to base units 220. In some embodiments, main base unit 210 is configured to act a centralized hub for the plurality of base units 220. In some embodiments, main base unit 210 is configured to provide electrical charging capability to client devices 110. In various embodiments, main base unit 210 is configured to communicate alerts triggered by the plurality of base units 220 to client devices 110. In some embodiments, main base unit 210 is configured to communicate within a network or with the internet. Although one main base unit is depicted, it should be appreciated that system 100 may include a plurality of main base units 210 and/or one of the plurality of base units 220 may act the main base unit 210.

As previously discussed, client device 110 may be a wearable device that identifies alerts to a user of client device 110 triggered by base unit 220. In some embodiments, base unit 220 is configured to alert client device 110 when motion is detected, a predetermined audible decibel/volume is detected, and/or a predetermined audible phrase is detected. One of ordinary skill in the art would understand that the alerts may be triggered by a variety of different detection activities and the examples described above are not intended to be limiting. In embodiments where client device 110 is a wearable device, it should be appreciated that the vibration of the wearable device permits activities to be performed that do not require constant audio monitoring such as working out with music, mowing the lawn, watching a loud movie, etc. Additionally, the range for detecting alerts identified by the various base unit 220 detection devices is increased because the wearable device has greater portability than traditional monitors.

In various embodiments, system 100 is configured to complement a monitoring system. In some embodiments, when the client device 110 is a wearable device, it should be appreciated that the wearable device is discreet. For example, the wearable device may be a discreet band that alerts the user wearing the band (and only that user) to an audible alert occurring in the room by vibrating. Advantageously, when client device 110 is a wearable device, the present disclosure may complement traditional monitors by alleviating frustrations that would impede traditional monitors (e.g., baby monitors) from functioning such as surrounding background noise or when the user is not co-located with the monitor. According to some embodiments, client device 110 is configured to allow users with third party client devices 110 (i.e., wearable devices, smart phones, etc.) to connect to base unit 220 and experience the same vibrating functionality and base identification as the client device 110 in wearable device embodiments. In this example, a user may also program the base unit 220 and/or client device 110 to receive specific vocal commands or respond to a specific audible level. Specific vocal commands may include programmable phrases and or preprogrammed audible decibel/volume levels.

System 100 disclosed herein may advantageously assist parents who would like to monitor the noise level, mainly during times of sleep, of their children. Specifically, at the appropriate time, parents would prefer to utilize a vibration to alert them of noise, rather than a traditional baby monitor. The following disclosure is also beneficial to the hearing impaired.

In various embodiments, client device 110 will have basic features of a chronograph watch, namely time and date. In various embodiments, to include when client device 110 is a wearable device, the wearable device may be actuated by a noise occurring in multiple spaces and display the space where the noise is occurring respectively. In this embodiment, if a noise occurs in a specific space, the wearable device will vibrate and display the alert received from the base unit 220, where the alert is generated from, and/or the decibel/volume level that triggered the alert. In some embodiments, client device 110 is a wearable device configured to illuminate upon receipt of the alert associated with the audible decibel/volume level based on the strength of the noise. In this embodiment, the strength of the illumination is associated with the strength of the audible decibel/volume level detected by base unit 220. In various embodiments, base unit 220 is configured to detect a decibel level via a microphone or similar audible detection means. In his embodiment, base unit 220 may have a decibel reader imbedded into the unit and may connect into a wall socket in the space or room where the audible decibel/volume level is intended to be identified. As described above and in a non-limiting manner here, base unit 220 may be configured to communicate via WiFi, Bluetooth, and/or any other wireless communication known to one of ordinary skill in the art. In various embodiments, main base unit 210 and/or base unit 220 is configured to charge client device 110 via USB, wired connection, or wirelessly charged. In some embodiments, client device 110 is a wearable device that may be charged at main base unit 210 and/or base unit 220.

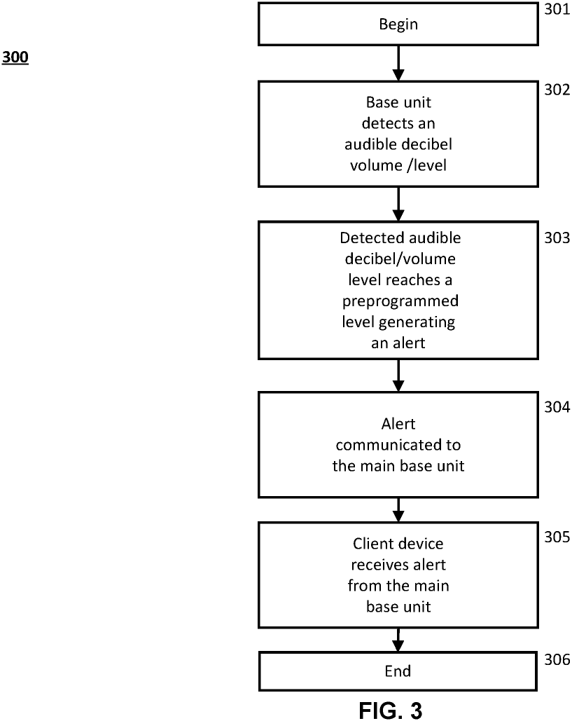

FIG. 3 is a flow diagram illustrating an example detection method 300 in accordance with some embodiments of the present disclosure. At 301, the method begins. At 302, base unit 220 detects an audible decibel/volume level. At 303, when the detected audible decibel/volume level reaches a preprogrammed level base unit 220 generates an alert. At 304, the alert generated by base unit 220 is communicated to client device 110 via main base unit 220. At 305, client device 110 receives the alert generated by base unit 220 via main base unit 220. At 306, the method ends .

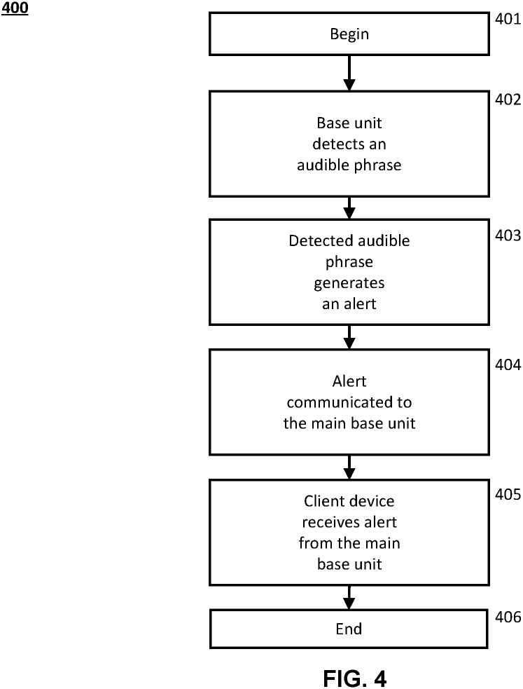

FIG. 4 is a flow diagram illustrating an example detection method 400 in accordance with some embodiments of the present disclosure. At 401, the method begins. At 402, base unit 220 detect an audible phrase. For example, base unit 220 may detect the phrase “Help 911.” In this example, main base unit 210 may be configured to receive programmable instructions relating to the audible phrase “Help 911” via client device 110. In various embodiments, main base unit 210 and/or base unit 220 may be programmed remotely or at the device to detect audible phrases created by the user. One of ordinary skill in the art would understand that base units 220 may passively listening to audio and generate an alert based on the audible decibel/volume level and/or an audible phrase. It should be appreciated that an audible phrase may be customized by the user to generate the alert communicated to client device 110. In various embodiments, client device 110 may be further programmed to perform specific functions upon receiving an alert. For example, the audible phrase “Help 911” may generate an alert on a plurality of client devices. In some embodiments, the alert may include a message, vibration, illumination, voice message, voice call, etc. One of ordinary skill in the art would understand that an alert as described herein may refer to a plurality of ways to alert a user with information relating to specific condition. At 403, when base unit 220 detects the audible phrase an alert is generated. At 404, the alert generated by base unit 220 is communicated to client device 110 via main base unit 220. At 405, client device 110 receives the alert generated by base unit 220 via main base unit 220. At 406, the method ends

In various embodiments, system 100 is configured to generate an alert to client devices 110 associated with motion or specific hand gesture. In some embodiments, base unit 220 may detect a general or specific motion in a space. In this embodiment, an alert may be generated at client device 110 associated with the general or specific motion. For example, base unit 220 may detect a specific hand gesture such as a waving hand or detect motion associated with a seizure. In this example, base unit 220 would generate an alert associated with the motion detected and communicate the alert to client device 110. In some embodiments, system 100 is configured to communicate with other devices or networks. For example, system 100 may be configured to contact emergency services upon the detection of a seizure by base unit 220 monitoring motion in a specific space within a house or building. A space as used herein may refer to any environment that may be monitored within a home, building, dwelling, or structure.

In some embodiments, system 100 is configured to generate an alert to client devices 110 associated with the lack of motion. For example, a user may identify a predetermined time period where a lack of movement in a space triggers an alert to client devices 110. In this example, the lack of movement will generate an alert or message indicating that no motion has occurred. In various embodiments, the lack of motion over a specific time period may be aggregated to generate an activity report. In this embodiment, the lack of motion over a specific period of time will be stored by system 100 and generate an activity report for a user of client device 110. Advantageously, this embodiment will be useful to track the decline of motion in elderly individuals over a period of time which is indicative of health. Alternatively, this embodiment could alert an individual of distress, health decline, or any reasonable physical condition that could be identified through the lack of movement.

In various embodiments, main base unit 210 and/or base unit 220 is configured to be registered. In this embodiment, registration may occur via an application or website accessible via client device 110 or computers 34, 54. In some embodiments, each component of system 100 to include main base unit 210, base unit 220, and/or client device 110 may be registered and/or associated with a particular user account. In this example, the user account may be associated with a particular home, building, dwelling or structure such that all devices are associated with the same monitoring system for a plurality of spaces. In some embodiments, registration may include an authentication method and identification associated with a particular space.

The present disclosure can be embodied in the form of methods and apparatus for practicing those methods. The present disclosure can also be embodied in the form of program code embodied in tangible media, such as secure digital (“SD”) cards, USB flash drives, diskettes, CD-ROMs, DVD-ROMs, Blu-ray disks, hard drives, or any other non-transitory machine-readable storage medium, wherein, when the program code is loaded into and executed by a machine, such as a computer, the machine becomes an apparatus for practicing the disclosure. The present disclosure can also be embodied in the form of program code, for example, whether stored in a storage medium, loaded into and/or executed by a machine, or transmitted over some transmission medium, such as over electrical wiring or cabling, through fiber optics, or via electromagnetic radiation, wherein, when the program code is loaded into and executed by a machine, such as a computer, the machine becomes an apparatus for practicing the disclosure. When implemented on a general-purpose processor, the program code segments combine with the processor to provide a unique device that operates analogously to specific logic circuits.

It may be emphasized that the above-described embodiments, are merely possible examples of implementations, and merely set forth a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and the present disclosure and protected by the following claims.

While this specification contains many specifics, these should not be construed as limitations on the scope of any disclosure or of what may be claimed, but rather as descriptions of features that may be specific to particular embodiments of particular disclosures. Certain features that are described in this specification in the context of separate embodiments may also be implemented in combination in a single embodiment. Conversely, various features that are described in the context of a single embodiment may also be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

While various embodiments have been described, it is to be understood that the embodiments described are illustrative only and that the scope of the subject matter is to be accorded a full range of equivalents, many variations and modifications naturally occurring to those of skill in the art from a perusal hereof.

What is claimed is:

1. A system, comprising:
at least one detection base unit configured to detect a decibel level associated with at least one space in a building and generate an alert when a predetermined decibel level is detected;
at least one main base unit configured to be communicably coupled to the at least one detection base unit and wirelessly receive and transmit the generated alert;
at least one client device configured to receive the transmitted alert and identify information associated with alert relating to the space within the building that corresponds to the associated detection base unit that generated the alert.

2. The system of claim 1, wherein the at least one client device is a wearable device.

3. The system of claim 2, wherein the wearable device vibrates upon receipt of the alert.

4. The system of claim 3, wherein the wearable device displays information related to the alert generated by the associated detection base unit.

5. The system of claim 1, wherein the at least one detection base unit is further configured to generate an alert upon the detection a preprogrammed audible phrase.

6. The system of claim 5, wherein the at least one main base unit is further configured to perform preprogrammed actions associated with the audible phrase.

7. The system of claim 6, wherein the preprogrammed actions include at least one of sending a text message, making a phone call, transmitting the alert to an external device, or alerting the client device.

8. The system of claim 1, wherein the main base unit is further configured to charge the client device.

9. A method, comprising:
detecting a decibel level by at least one detection base unit associated with at least one space in a building;
generating an alert when a predetermined decibel level is detected;
receiving the generated alert wirelessly by at least one main base unit;
transmitting the generated alert from the at least one main base unit to at least one client device;
identifying information associated with generated alert at the client device associated with the space within the building that corresponds to the respective detection base unit that generated alert.

10. The method of claim 9, wherein the at least one client device is a wearable device.

11. The method of claim 10, wherein the wearable device vibrates upon receiving the generated alert.

12. The method of claim 11, wherein the wearable device displays information related to the alert generated by the associated detection base unit.

13. The method of claim 9, further comprising the steps of generating an alert upon detecting a preprogrammed audible phrase.

14. The method of claim 14, wherein the at least one main base unit is configured to perform preprogrammed actions associated with the audible phrase.

15. The method of claim 15, wherein the preprogrammed actions include at least one of sending a text message, making a phone call, transmitting the alert to an external device, or alerting the client device.

16. The method claim 9, wherein the main base unit is configured to charge the client device.

17. A system, comprising:
at least one detection base unit configured to detect sounds associated with at least one space in a building and generate an alert when either a predetermined decibel level or preprogrammed audio phrase is detected;
at least one main base unit configured to be communicably coupled to the at least one detection base unit and wirelessly receive and transmit the generated alert;
at least one client device configured to receive the transmitted alert and identify information associated with alert relating to the space within the building that corresponds to the associated detection base unit that generated the alert.

18. The system of claim 17, wherein the at least one detection unit is further configured to detect motion associated with at least one space in a building and generate an alert when detected.

19. The system of claim 18, wherein the motion is a preprogrammed gesture.

20. The system of claim 19, wherein the client device is a wearable device.
